(19) United States
(12) Patent Application Publication
(10) Pub. No.: US 2011/0008899 A1
(43) Pub. Date: Jan. 13, 2011

(54) APPARATUS FOR DETERMINING THE COMPOSITION OF A SPECIMEN, IN PARTICULAR ONE CONTAINING PROTEIN

(76) Inventor: Yiannakis Polemitis, Neuss (DE)

Correspondence Address: CLARK & BRODY, 1700 Diagonal Road, Suite 510, Alexandria, VA, 22314, US

(21) Appl. No.: 12/557,935

(22) Filed: Sep. 11, 2009

(30) Foreign Application Priority Data
Jul. 13, 2009 (EP) 09 009 131.5

(51) Int. Cl. G01N 33/68; G01N 33/00

(52) U.S. Cl. 436/86; 422/83

(57) ABSTRACT

An apparatus and a method for determining the composition of a protein-containing specimen has a combustion chamber for the combustion of the specimen, an exhaust gas line connected to a combustion chamber heated by a heating device, a combustion control device, an oxygen inflow line opening into the combustion chamber with an oxygen inflow valve, which can be operated by means of the control device, and an analyser connected to the combustion chamber. An oxygen sensor connected to the control device determines the oxygen content in the combustion gases. The oxygen content is determined in time intervals or continuously during combustion. If a pre-specifiable concentration of oxygen is not achieved, a definable quantity of oxygen is injected into the combustion chamber by the oxygen inflow valve. After complete combustion of the specimen, the combustion gases are delivered to the analyser to determine nitrogen, carbon dioxide, sulphur dioxide and/or water content.

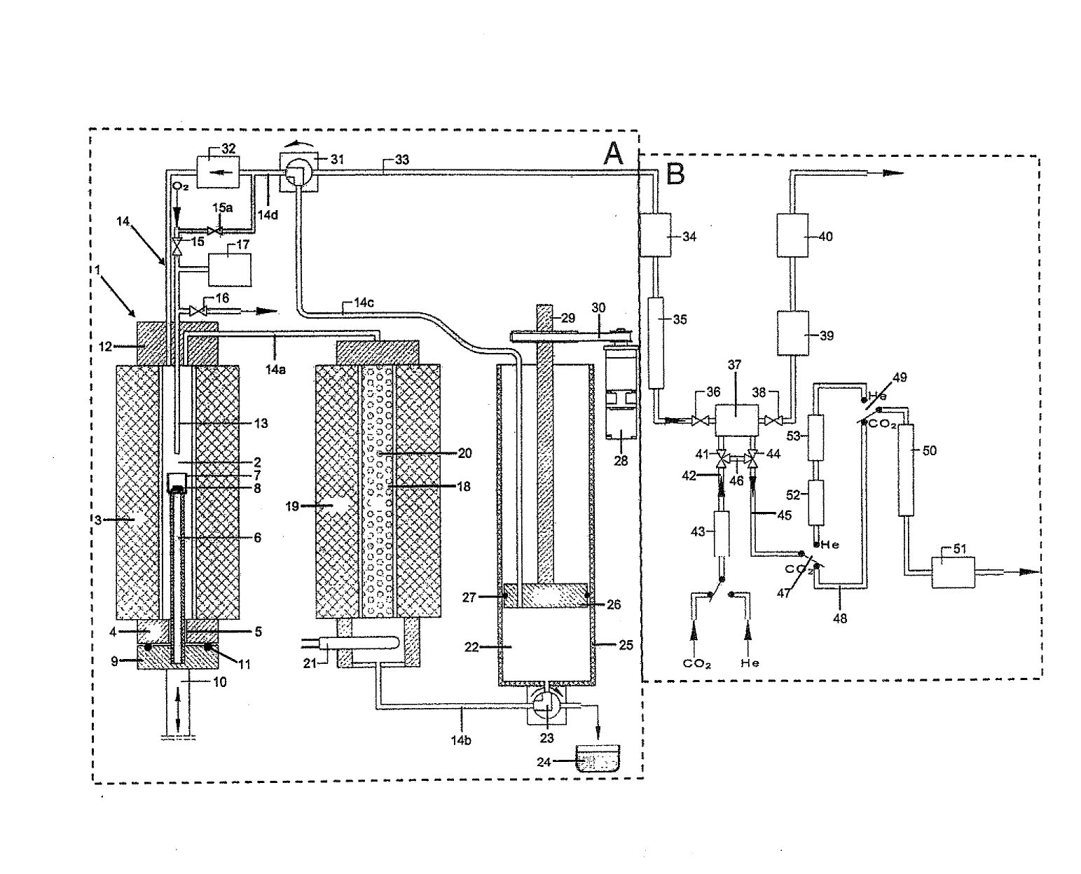

An apparatus for determining the composition of a specimen, in particular one containing protein

The present invention relates to an apparatus for determining the composition of a specimen, in particular one containing protein, having a combustion chamber for the combustion of the specimen to be analysed, an exhaust gas line connected to the combustion chamber, a heating device for heating the combustion chamber, and a control device for controlling the combustion, an oxygen inflow line opening out into the combustion chamber and having an oxygen inflow valve which can be operated by means of the control device, and an analyser being connected to the combustion chamber. The invention further relates to a method of determining the composition of a specimen, in particular one containing protein, with the aid of the apparatus according to the invention.

Analysis apparatuses of this type for determining the composition from the combustion products of materials to be analysed are known, for example, from DE 33 019 71 A1. A U-shaped analysis oven is described here with which the carbon, hydrogen and nitrogen content of an organic specimen can be determined after combustion of the latter. For this purpose the specimen is poured into a tin capsule and positioned on a specimen holder which is located within a U-shaped combustion tube. The combustion tube is heated to 1000° C., and the specimen is subjected to a flow of oxygen from above by means of a lance, by means of which the specimen combusts. The gases produced during combustion are subjected to different separation steps, the dust produced by the tin capsule, which also combusts in this process, being filtered off. The combustion gases are delivered to the analyser by continuously introducing oxygen into the combustion chamber.

This type of apparatus is associated with various disadvantages. On the one hand the continuous supply of oxygen and the use of the latter as a gas for conveying the combustion gases to the analyser leads to significant consumption of this gas. Since for the analysis the oxygen must be removed again reductively, as is generally implemented with the aid of a copper granulate, the method is expensive, not least also due to the one-way tin capsules to be used with this method.

A further disadvantage results from the use of tin capsules for the combustion because under the combustion conditions of the analysis oven the latter combust to form tin oxide dust which contaminates the interior of the oven and necessitates the use of a dust filter in order to protect the sensitive detectors from the tin oxide dust. A further difficulty arises with this type of analysis oven due to the fact that, in particular with specimens with a small portion of nitrogen, the precise quantitative measurement of the nitrogen content is problematic in the presence of high oxygen concentrations from the carrier gas. Moreover, it can be considered as partially disadvantageous that with this type of apparatus only relatively small specimen quantities, generally up to orders of magnitude of approximately 100 mg, can be used. With strongly unhomogeneous specimens such as carbon, flour, meat, animal feed etc., this may lead to unrepresentative measurement results. In every case the preparation of the specimen with such unhomogeneous specimens is difficult and time-consuming.

In EP 1 207 390 an analysis oven is described with which, even with small quantities of nitrogen, the determination of the nitrogen content of a specimen is itself possible in the presence of relatively high concentrations of oxygen. For this purpose the gas mixture produced during the combustion of the specimen is passed via a washing device in which the combustion gas is freed as far as possible from oxygen. The remaining components of the combustion gas are then investigated by means of a sensor for their nitrogen content. However, the removal of large quantities of oxygen requires complex apparatus and the high oxygen requirement of this apparatus leads to an increase in analysis costs.

WO 2006/074720 describes an analysis oven with which combustion gases produced during the combustion of a specimen to be investigated are separated into their N₂, CO₂, H₂O and SO₂ components by means of adsorption traps arranged in a series. These adsorption traps are designed such that they selectively respectively adsorb an individual combustion product. In the subsequent analysis step the adsorption traps are heated individually one after the other by means of which the adsorbed combustion gases are desorbed and can be subjected to a quantitative analysis. This procedure requires a complex apparatus layout since for each component to be analysed, in addition to a sensitive detector for this purpose, a sufficiently large adsorption volume must also be produced. Moreover, the precise setting of the individual adsorption conditions can be difficult.

The object of the present invention consists of designing an apparatus for determining the composition of a specimen, in particular one containing protein, which allows the introduction of a wide variety of specimen quantities, and in particular also large specimen quantities, and thus achieves total combustion of the specimen material with the smallest possible quantity of oxygen.

This object is achieved by an apparatus and a method of the type specified at the start wherein an oxygen sensor for determining the oxygen content in the combustion gases is provided and is connected to the control device, and the apparatus is designed such that the oxygen content in the combustion gases is determined by means of the oxygen gas sensor at intervals of time or continuously during the combustion of the specimen, and if a pre-specifiable oxygen concentration is not achieved, a definable quantity of oxygen is injected into the combustion chamber by means of the oxygen inflow valve, and after complete combustion of the specimen the combustion gases are delivered to the analyser where, in particular, the nitrogen and/or carbon dioxide and/or sulphur dioxide and/or water content in the combustion gases is determined. In this way the nitrogen, hydrogen, carbon and/or sulphur content of the specimen can be determined with the apparatus according to the invention. The achievable detection limit for nitrogen is in particular 0.01% by weight nitrogen in the specimen to be analysed.

The apparatus according to the invention enables complete combustion of the specimen to be analysed with the smallest possible quantity of oxygen. For this purpose, for combustion the specimen is first of all converted with a substoichiometric quantity of oxygen, and the oxygen content is determined during the combustion process. If a pre-specified minimum content of oxygen is not achieved, a defined quantity of oxygen is metered into the combustion chamber. The oxygen content is continuously checked during the whole combustion, and so long, successive oxygen administrations are undertaken until one no longer fails to achieve the pre-specified minimum oxygen content.

In this way very different specimen quantities can be analysed without any adaptation of the apparatus being required for this. Since the consumption of oxygen in relation to the specimen mass is strongly dependent upon the nature of the specimen, in this regard too the apparatus according to the invention offers a very high degree of flexibility. Furthermore, the apparatus according to the invention allows the use of recyclable ceramic crucibles instead of the previously conventional one-way capsules made of tin.

Completeness of combustion can be considered as having been achieved, for example, if one no longer fails to achieve the pre-specifiable oxygen concentration over a pre-specified period of time and/or the reduction in oxygen concentration no longer exceeds a pre-specified temporal change. This can be, for example, approximately 1 to 2 minutes. Even if these two methods have turned out to be particularly practicable, other criteria can also be used in order to establish the time of the complete combustion of the specimen.

The oxygen concentration pre-specifiable for the triggering of oxygen administration, and which if not achieved oxygen is delivered to the combustion chamber, can be set to any values. Particularly low limit values for the oxygen concentration lead to particularly low increased consumption of oxygen, but at the same time increase the duration of the analysis. Conversely, the specification of relatively high limit values for the oxygen concentration leads to a relatively fast analysis with, however, potentially greater oxygen consumption. In this context greater oxygen consumption is understood as being the quantity of oxygen which is delivered to the combustion chamber, but is not converted for the combustion of the specimen because the state of complete combustion has already been achieved. Even if the person skilled in the art is free to choose the appropriate oxygen limit values, limit values in the range from 1% to 4% have turned out to be benchmarks.

The concept of complete combustion of the specimen is understood to mean that in their normal pressure under combustion conditions, for example 1000° C., the combustion products are in a thermodynamically stable form, i.e. carbon as CO₂, hydrogen as H₂O, nitrogen as NOₓ, i.e. NO and/or NO₂ and sulphur as SO₂.

According to a further development of the apparatus according to the invention, the exhaust gas line is in the form of a bypass line by means of which during combustion of the specimen the combustion gases are discharged from the combustion chamber and then conveyed back into the combustion chamber. In addition, the apparatus has a conveying device for conveying the combustion gases through the bypass line during combustion of the specimen. A circulation pump or a compressor, for example, can be considered as a conveying device. In a further embodiment the oxygen sensor is connected to the bypass line. The circulation of the combustion gases through the circulation pump or the compressor contributes to the completeness of combustion and leads, moreover, to homogenisation of the combustion products and of the oxygen contained in the available volume of gas which is circulated by the circulation pump or the compressor. In this way more precise determination of the oxygen content is also achieved by means of the oxygen sensor.

In the exhaust gas line a post-combustion chamber can be provided downstream of the combustion chamber and to which a heating device is assigned. The post-combustion chamber ensures that specimen components from the combustion chamber which have not completely combusted are subjected to post-combustion. In order to improve the post-combustion the post-combustion chamber can be equipped, for example, with ceramic balls, ceramic sand and/or an oxidation catalyst. These fillers have the effect, moreover, of homogenising the combustion gases. A platinum/rhodium catalyst or other oxidation catalysts known from the automotive domain, for example, are used as an oxidation catalyst.

With this embodiment the oxygen sensor is advantageously disposed on the outlet of the post-combustion chamber because at this point the combustion gas/oxygen mixture is relatively well homogenised. The oxygen sensor can be in the form of an oxygen electrode, in particular as a lambda sensor. These types of oxygen sensor provide reliable measurement results, even with high ambient temperatures.

The combustion chamber and the post-combustion chamber can be heated independently of one another to the temperature generally required for the combustion of a specimen in a pure oxygen atmosphere. These can be temperatures, for example, of 800 to 1200° C., preferably approximately 1000° C. The combustion gases are circulated by means of a conveying device until there is complete combustion of the specimen by being delivered from the outlet side of the post-combustion chamber back to the combustion chamber.

Preferably, the combustion of the specimen is implemented as far as possible isobarically, i.e. with constant pressure. Since, however, exhaust gases are produced with the combustion of the specimen, the volume of gas within the apparatus increases during operation. In order, nevertheless, to keep the pressure constant within the apparatus, which is sealed off from the surroundings, a pressure compensation chamber with a changeable volume is preferably provided. This pressure compensation chamber is preferably connected to the exhaust gas line passing out of the post-combustion chamber, if the latter is indeed provided, and can be realised by any device with which the volume can be set variably. Within the framework of the present invention an embodiment is preferred wherein in the exhaust gas line a pressure compensation chamber is provided which is formed between a pressure compensation vessel and a piston moveably guided therein by means of a positioning member, by adjusting the piston the pressure within the apparatus being set and/or it being possible for oxygen to be sucked into the combustion chamber via the oxygen inflow line and/or it being possible for the combustion gases to be delivered to the analyser. The delivery of the combustion gases to the analyser is implemented, for example, by means of an analyser line on which the section passing away from the pressure compensation chamber is coupled to the exhaust gas line by means of a two-way valve.

The positioning member can be driven by a motor, it being possible for control of the motor to be taken over by the control device. Moreover, the positioning member can be connected to a pressure sensor for measuring the pressure within the apparatus. In this way particularly precise setting and maintenance of the desired pressure is possible.

Therefore, a pressure compensation chamber designed in this way fulfils at least three different functions. On the one hand, in combination with a pressure sensor the pressure within the apparatus can be set to a desired level. It is therefore not necessary to design the apparatus to be pressure-resistant, as is the case, for example, with autoclaves. The further function fulfilled by the pressure compensation chamber is the precise metering of oxygen. By means of the positioning member the piston can be moved within the pressure compensation chamber by very small increments, by means of which precisely settable oxygen volumes can be sucked into the combustion chamber. Finally, by adjusting the piston the combustion gases located within the pressure compensation chamber can be delivered to the analyser.

According to a further embodiment of the apparatus according to the invention the section of the exhaust gas line leading away from the pressure compensation chamber passes through the piston and is preferably entrained with an adjustment of the piston. The integration of the exhaust gas line into the piston ensures that the pressure compensation chamber can fulfill its function in any position of the piston, in particular if the exhaust gases are delivered through the floor of the pressure compensation vessel. Moreover, with this type of arrangement the delivery and discharge of the combustion gases are spatially separated so that additionally a further homogenisation of the combustion gases with the oxygen contained in the gas atmosphere can take place in the pressure compensation chamber. Furthermore, this makes it possible to withdraw any water condensation which may have formed on the bottom of the pressure compensation vessel. For this purpose the apparatus can have at least one water separator, it being preferable if the quantity of water separated can be determined.

With the aid of the apparatus according to the invention the specimen to be analysed can be investigated with regard to its nitrogen, carbon, sulphur and/or hydrogen content. For this purpose the analyser preferably comprises at least one nitrogen and/or one carbon dioxide and/or one sulphur dioxide and/or one humidity sensor. With the exception of the sensor for determining nitrogen, these sensors are, for example, in the form of infrared detectors. The sensor for determining nitrogen can be realised by means of a heat conductivity sensor, even if naturally other appropriate sensors can also be used. The delivery of the combustion gases to the carbon dioxide, humidity, sulphur dioxide and/or sulphur trioxide sensor is preferably implemented by adjusting the piston in the pressure compensation vessel. By this type of delivery of the combustion gases one can at this point dispense with a carrier gas, and this is associated with cost advantages. Moreover, in this way no ballast gases which could possibly interfere with the measurements are fed in.

For more target-orientated delivery of the oxygen to the specimen to be combusted said oxygen can be conveyed by means of an oxygen lance coupled to the oxygen inflow line to the vicinity of the specimen to be combusted. The specimen can be attached onto a specimen holder which comprises in particular a ceramic crucible, and is preferably introduced from below into the combustion chamber. Unlike the conventionally used tin crucibles, ceramic crucibles are characterised in that they can be used a number of times and do not lead to contamination of the apparatus.

In a further development of the apparatus according to the invention the analyser comprises at least one sensor for determining nitrogen upstream of which there is a gas specimen chamber for accommodating a defined volume of combustion gas. For this purpose the gas specimen chamber can be filled with combustion gases by adjusting a piston of a pressure compensation vessel after the specimen to be analysed is completely combusted. Furthermore, a carrier gas inflow line can open out into the gas specimen chamber via a through flow regulator. By means of this carrier gas inflow line the defined volume of combustion gas can be delivered to the sensor by means of a carrier gas, such as for example helium or carbon dioxide, for determining nitrogen.

For precise determination of the nitrogen content from this volume of combustion gas it is advantageous to remove components, such as for example oxygen, which interfere with the measurement from the combustion gases. This can be implemented by means of an oxygen trap, known in its own right, which oxidatively binds the oxygen contained in the combustion gases. At the same time, for the most precise possible determination of the nitrogen content, the nitrogen oxides contained in the combustion exhaust gases should be converted into molecular nitrogen (N₂). For this purpose the volume of combustion gas is conveyed over a nitrogen oxide reduction catalyst. These two functions can be implemented either in two different devices or also in one unit. A heated copper, molybdenum or wolfram catalyst fulfils for example both requirements. These catalyst materials are used, for example, in the form of fine-mesh nets or as granulates and heated to a temperature of 700 to 800° C.

The values for nitrogen, carbon, sulphur and hydrogen determined by the individual sensors can be converted by the specimen weight into percentages by weight of these elements of the composition of the specimen.

In the drawing the invention is illustrated in greater detail by means of an exemplary embodiment.

In the single figure an apparatus according to the invention for determining composition with a combustion unit A and an analyser B are shown. The combustion unit A has a combustion device 1 having a combustion chamber 2 and a heating device 3 in the form of an electric resistance heating system enclosing the latter. The combustion device 1 has on its lower side a stop surface 4 which has an opening 5 through which a specimen holder in the form of a crucible carrier 6 is passed on which a crucible 7 made of ceramic with a specimen 8 to be analysed is fixed. The crucible carrier 6 is attached to a shaft 10 moveable by means of a vertical pneumatic drive and which has a stop surface 9. Disposed between the stop surfaces 4 and 9 is a sealing ring 11 which on the lower side closes off the combustion chamber 2 from the surroundings, gas-tight.

The open upper side of the combustion chamber 2 is closed by a cover 12 through which an oxygen inflow line 13 passes. The oxygen inflow line 13 is in the form of an oxygen lance the outlet of which is located directly over the specimen 8 to be analysed. The oxygen inflow line 13 is connected to an oxygen source (not shown here) from which oxygen is metered via an oxygen inflow valve 15 provided in the oxygen inflow line 13 into the combustion chamber. The oxygen inflow valve 15 is coupled to a control device (not shown). Coupled to the oxygen inflow line 13 is an excess outlet valve 16 via which, for example, excess oxygen can be released into the surroundings. With the aid of a pressure sensor 17 connected to the oxygen inflow line 13 the internal pressure in the apparatus, in particular in the region of the combustion unit A, can be measured. The pressure sensor 17 is connected to the control device.

The exhaust gas line in the form of a bypass line 14 comprises a number of sections 14a-d by means of which the combustion gases are led away from the combustion chamber 2 via the exhaust gas line section 14a and conveyed back to the latter after completion of the cycle via the exhaust gas line section 14d. Moreover, the exhaust gas line section 14d is connected by means of a valve 15a to the oxygen source. Adjacent to this the exhaust gas line sections 14a and 14d respectively pass through the cover 12 of the combustion chamber 2.

Starting from the combustion chamber 2, the exhaust gas line section 14a ends in a post-combustion chamber 18 which is surrounded by a heating device 19 in the form of an electric resistance heating system and is filled with a catalyst 20. Adjoining the post-combustion chamber 18 on the outlet side is an oxygen sensor connected to the control device in the form of a lambda sensor 21 and the exhaust gas line section 14b which opens out into a pressure compensation chamber 22. Integrated into the exhaust gas line section 14b is a water separation valve 23 by means of which any condensed water formed can be discharged into a water separator 24.

The pressure compensation chamber 22 is formed between a pressure compensation vessel 25 and a piston 26 moved within the latter, torsion-proof, and sealed off from the surroundings by a piston ring 27. The piston 26 is held such as to be vertically adjustable by a drive 28 connected to the control device 28 by means of a positioning member in the form of a threaded rod 29, the transmission of power from the drive 28 to the threaded rod 29 being implemented by means of a toothed belt 30.

The exhaust gas line section 14c passing from the pressure compensation chamber 22 passes through the piston 26 and is designed such that it is entrained by an adjustment of the piston 26. It leads to a two-way valve from which the exhaust gas line section 14d is conveyed back through the cover 12 into the combustion chamber 2. A conveying device in the form of a circulation pump 32 is integrated into the exhaust gas line section 14d. In addition to the exhaust gas line sections 14c and 14d adjoining the two-way valve 31 is an analyser line 33 which leads from the combustion unit A to the analyser B.

The analyser line 33 leads via a humidity/H₂O infrared detector 34 and a water absorber 35 and opens out adjustably via a valve 36 into a gas specimen chamber 37. The latter is connected separably to a CO₂ infrared detector 39 and a SO₂ infrared detector 40 by means of a valve 38.

A carrier gas inflow line 42 connected to a carrier gas source leads via a through flow regulator 43 and a valve 41, blockably, into the gas specimen chamber 37, the carrier gas source optionally supplying CO₂ and/or helium. A carrier gas outflow line 45 leads away from the gas specimen chamber 37 via a valve 44, a bypass 46 being located between the valves 41 and 44.

The carrier gas outflow line 45 leads via a valve 47 to a line 48 with which a combined oxygen trap/nitrogen oxide reduction means 50 is connected by means of a valve 49 to heatable copper granulate to which a heat conductivity detector adjoins 51. Disposed parallel to the line 48, between the valves 47 and 49, is a carbon dioxide absorber 52 and a water absorber 53 for the event that helium is chosen as a carrier gas.

In the following the method procedure during operation of the apparatus illustrated in the figure is described. The specimen 8 to be analysed is weighed in the crucible 7, and the latter is fixed 6 onto the crucible carrier 6.

The crucible carrier 6 is located here beneath the combustion chamber 2 because the shaft 10 of the pneumatic drive is located in its lowermost vertical position.

The valve 15a is opened, and by means of this the combustion chamber 2, the bypass line 14, the post-combustion chamber 18 and the pressure compensation chamber 22 are totally flooded with oxygen. By briefly opening the oxygen inflow valve 15 the oxygen inflow line 13 is flushed with oxygen. The circulation pump 32 is switched to a low output and so is also filled with oxygen. The water separation valve 23 is closed, and the two-way valve 31 connects the exhaust gas line sections 14c and 14d, by means of which the analyser B is uncoupled.

After the total interior and the gas conveyance system are flooded with oxygen and an existing excess has flowed out of the combustion chamber 2 through the opening 5 located in the stop surface 4, the specimen is introduced into the combustion chamber 2 by means of the pneumatically driven shaft 10 until the stop surface 9 runs against the stop surface 4 and the combustion chamber 2 is sealed off by the sealing ring 11. The oxygen inflow valve 15 is closed, and the combustion chamber 2 and the post-combustion chamber 18 are heated to approx. 1000° C. by the heating devices 3 and 19, by means of which the specimen 8 combusts. During continuous operation the combustion chamber 2 and the post-combustion chamber 18 are kept permanently at this temperature. The combustion gases are conveyed by the circulation pump 32 via the exhaust gas line section 14a into the post-combustion chamber 18 where, supported by the catalyst 20, post-combustion of not completely combusted components of the specimen 8 takes place.

The oxygen content of the combustion gas/oxygen mixture is measured behind the post-combustion chamber 18 by the lambda sensor 21, the measurement signals being forwarded to the control device. If a pre-specified oxygen minimum concentration is exceeded, the oxygen inflow valve 15 and the excess outlet valve 16 are opened by the control device, and the piston 26 is pulled upwards by a pre-specified distance by the drive 28 by means of which the volume of the pressure compensation chamber 22 is increased and oxygen is sucked via the now open oxygen inflow valve 15 and the oxygen lance 13 into the combustion chamber 2. Here the latter provides further combustion of the specimen 8.

After the volume of the pressure compensation chamber 22 has been increased by the desired amount, the oxygen inflow valve 15 and the excess outlet valve 16 are closed, and the combustion gas/oxygen mixture is further continuously circulated by the circulation pump 32 through the combustion chamber 2, the post-combustion chamber 18, the pressure compensation chamber 22 and the exhaust gas line 14.

During the overall combustion process the internal pressure in this system is checked by means of the pressure sensor 17. If a pre-specified value is exceeded or fallen short of, the volume of the pressure compensation chamber 22 is changed by the control device by means of the drive 28 such that the pre-specified internal pressure is re-set. Moreover, during the combustion process the detectors 34, 39 and 40 can be calibrated. For this purpose, on the analyser line 33 an oxygen flushing valve, not shown for reasons relating to simplification, can be provided by means of which the oxygen can be conveyed into the analyser line 33 and floods the humidity detector 34, the water absorber 35, the gas specimen chamber 37, the CO₂ detector 39 and the SO₂ detector 40 with oxygen. The detectors 34, 39 and 40 filled with oxygen can now be calibrated to their zero line. Before the combustion gases are conveyed into the analyser line 33, the oxygen flushing valve is closed and the oxygen flushing process of the detectors 34, 39 and 40 is therefore stopped.

This process, comprising determining the oxygen content and adding a defined quantity of oxygen if a minimum concentration of oxygen is not achieved, is implemented until the minimum concentration of oxygen is no longer fallen short of, for example over a period of approximately 1 to 2 minutes. In this state the specimen 8 is completely combusted.

After the combustion process has ended the circulation pump 32 is stopped and the two-way valve 31 is reversed, by means of which the bypass line 14 is interrupted and the exhaust gas line section 14c is connected to the analyser line 33. With the aid of the drive 28 the piston 26 is moved into the pressure compensation vessel 25 in order to reduce the pressure compensation chamber 22, by means of which the combustion gases flow via the exhaust gas line section 14d, the two-way valve 31 and the analyser line 33 through the humidity detector 34. From the measurement signal of the humidity detector 34 the percentage water content in the combustion gases can be determined from which in turn the proportion of hydrogen in the specimen 8 can be calculated.

The combustion gases are conveyed via the water absorber 35, are completely dried here and then flow via the opened valves 36 and 38 through the gas specimen chamber 37, the valves 41 and 44 being closed in relation to the gas specimen chamber 37. The gas specimen chamber 37 is completely flooded here with the combustion gases. By means of the open valve 44 the combustion gases are conveyed via the CO₂ detector 39 and the SO₂ detector 40 which determine the percentage carbon dioxide and sulphur dioxide SO₂ content in the combustion gases. One can conclude the carbon content and the sulphur content of the specimen 8 from the values established.

During this analysis procedure a carrier gas, for example helium, flows via the through flow regulator 43 through the carrier gas inflow line 42, the valve 41, the bypass 46 and the valve 44 into the carrier gas outflow line 45. The valves 36 and 38 are then closed and the valves 41 and 44 activated so that the bypass line 46 is closed and the line 42 and 45 are connected to the gas specimen chamber 37. The stream of helium therefore flows via the carrier gas inflow line 42 into the gas specimen chamber and displaces the combustion gases here towards the carrier gas outflow line 45. On this path a mixture of carrier gas and combustion gases is conveyed via the valve 47 through the carbon dioxide absorber 52, the water absorber 53, the valve 49 and the combined oxygen trap/nitrogen reduction means 50.

If CO₂ is used as a carrier gas, the carrier gas/combustion gas mixture flows via the line 48 to the heated copper granulate 50 instead of via the carbon dioxide absorber 52 and the water absorber 53.

In the combined oxygen trap/nitrogen oxide reduction means 50 any possible residual oxygen is removed by oxidation of the copper granulate and the nitrogen oxides contained in the combustion gases are reduced to molecular nitrogen (N₂). The total nitrogen contained in the combustion gases is now present in the form of molecular nitrogen which is integrally determined by means of the heat conductivity detector 51. Since the volume of the gas specimen chamber 37 is known, in this way the percentage nitrogen content in the combustion gases can be determined, from which the nitrogen content of the specimen 8 can be calculated.

1. An apparatus for determining the composition of a specimen (8), in particular one containing protein, having a combustion chamber (2) for the combustion of the specimen (8) to be analysed, an exhaust gas line (14) connected to the combustion chamber (2), a heating device (3) for heating the combustion chamber (2) and a control device for controlling the combustion, an oxygen inflow line (13) opening out into the combustion chamber (2), and having an oxygen inflow valve (15) which can be operated by means of the control device, and an analyser (B) being connected to the combustion chamber (2), characterised in that an oxygen sensor (21) for determining the oxygen content in the combustion gases is provided, and is connected to the control device, and the apparatus is designed such that the oxygen content in the combustion gases is determined by means of the oxygen sensor (21) at intervals of time or continuously during combustion of the specimen (8), and if a pre-specifiable concentration of oxygen is not achieved, a definable quantity of oxygen is injected into the combustion chamber (2) by means of the oxygen inflow valve (15), and after complete combustion of the specimen (8) the combustion gases are delivered to the analyser (B) where, in particular, the nitrogen and/or carbon dioxide and/or sulphur dioxide and/or water content in the combustion gases is determined.

2. The apparatus according to claim 1, characterised in that the exhaust gas line is in the form of a bypass line (14) by means of which during combustion of the specimen (8) the combustion gases are discharged from the combustion chamber (2) and then conveyed back into the combustion chamber (2), and a conveying device, in particular a circulation pump (32) or a compressor is provided for conveying the combustion gases through the bypass line (14), the oxygen sensor (21) preferably being disposed on the bypass line (14).

3. The apparatus according to claim 1, characterised in that in the exhaust gas line (14) a post-combustion chamber (18) is provided downstream of the combustion chamber (2) to which a heating device (19) is assigned.

4. The apparatus according to claim 3, characterised in that the post-combustion chamber (18) is equipped with an oxidation catalyst (20), ceramic balls and/or ceramic sand and that the oxygen sensor (21) is preferably provided on the outlet side of the post-combustion chamber (18).

5. The apparatus according to claim 1, characterised in that in the exhaust gas line (14) a pressure compensation chamber (22) is provided preferably downstream of a post-combustion chamber (18) downstream of the combustion chamber (2) and which is formed between a pressure compensation vessel (25) and a piston moveably guided therein by means of a positioning member (29), by adjusting the piston (26) the pressure within the apparatus being set and/or it being possible for oxygen to be sucked into the combustion chamber (2) via the oxygen inflow line (13) and/or it being possible for the combustion gases to be delivered to the analyser (B).

6. The apparatus according to claim 5, characterised in that the positioning member (29) is connected to a pressure sensor (17) for measuring the pressure within the apparatus.

7. The apparatus according to claim 5, characterised in that the section (14c) of the exhaust gas line (14) leading away from the pressure compensation chamber (22) passes through the piston (26) and is preferably entrained with an adjustment of the piston (16).

8. The apparatus according to claim 1, characterised in that the analyser (B) comprises a sensor for determining nitrogen (51) upstream of which there is a gas specimen chamber (37) for accommodating a defined volume of combustion gas and that a carrier gas inflow line (42) opens out into the gas specimen chamber (37) via a through flow regulator (43).

9. A method of determining the composition of a specimen (8), in particular one containing protein, wherein the specimen (8) to be analysed is completely combusted in an oxygen atmosphere and the composition of the combustion gases produced is then determined, characterised in that the oxygen content in the combustion gases is determined by means of an oxygen sensor (21) at intervals of time or continuously during combustion of the specimen (8), and if a pre-specifiable concentration of oxygen is not achieved, a definable quantity of oxygen is delivered to the combustion, and that after complete combustion of the specimen (8) the resulting combustion gases are delivered to an analyser (B) where, in particular, the nitrogen and/or carbon dioxide and/or sulphur dioxide and/or water content in the combustion gases is determined.

10. The method according to claim 9, characterised in that completeness of combustion is considered to have been achieved if one no longer fails to achieve the pre-specifiable concentration of oxygen over a pre-specified period of time and/or the reduction of the oxygen concentration no longer exceeds a pre-specified temporal change.

11. The method according to claim 9, characterised in that the combustion of the specimen (8) is implemented in a heated combustion chamber (2) to which is connected an exhaust gas line in the form of a bypass line(14) through which the combustion gases are withdrawn from the combustion chamber (2) until the specimen (8) has been completely combusted and delivered back to the latter.

12. The method according to claim 11, characterised in that in the bypass line (14) a heated post-combustion chamber (18) is preferably provided in which the combustion gases coming out of the post-combustion chamber (2) are subjected to post-combustion.

13. The method according to claim 9, characterised in that the pressure in the apparatus is set by means of a pressure compensation chamber (22) connected by means of the exhaust gas line (14) and preferably disposed on the outlet side of a post-combustion chamber (18), which is formed between a pressure compensation vessel (25) and a piston (26) guided moveably therein by a positioning member (29), by adjustment of the piston (26) the pressure within the apparatus being set and/or oxygen being sucked into the combustion chamber (2) by means of the oxygen inflow line (13) and/or the combustion gases being delivered to the analyser (B).

14. The method according to claim 13, characterised in that the pressure within the apparatus is measured by means of a pressure sensor (17) and the positioning member (29) is operated such that a desired pressure is set.

15. The method according to claim 9, characterised in that the analyser (B) comprises a sensor for determining nitrogen (51) upstream of which there is a gas specimen chamber (37) with a defined volume of combustion gas and which is totally filled with combustion gases and then freed from oxygen, any nitrogen oxides contained being converted into nitrogen (N₂) and being delivered to the sensor for nitrogen determination (51).
